(19) United States
(12) Patent Application Publication
(10) Pub. No.: US 2022/0414884 A1
(43) Pub. Date: Dec. 29, 2022

(54) MEDICAL INFORMATION PROCESSING APPARATUS, MEDICAL INFORMATION PROCESSING METHOD, AND MEDICAL INFORMATION PROCESSING PROGRAM

(71) Applicant: Canon Medical Systems Corporation, Otawara-shi (JP)

(72) Inventor: Hidenori TAKESHIMA, Kawasaki (JP)

(73) Assignee: Canon Medical Systems Corporation, Otawara-shi (JP)

(21) Appl. No.: 17/806,353

(22) Filed: Jun. 10, 2022

(30) Foreign Application Priority Data
Jun. 25, 2021 (JP) 2021-105899

(51) Int. Cl. G06T 7/00; G16H 30/40

(52) U.S. Cl. CPC G06T 7/0016; G16H 30/40; G06T 2207/20081; G06T 2207/10088; G06T 2207/10081; G06T 2207/30004; G06T 2207/30168; G06T 2207/20084

(57) ABSTRACT

According to one embodiment, a medical information processing apparatus comprising processing circuitry. The processing circuitry acquires first medical data having a first data resolution and second medical data having a second data resolution. The processing circuitry acquires an acquisition condition for medical data relating to at least one of the first medical data and the second medical data. The processing circuitry outputs a result integrated at least a piece of information that is based on the first medical data and the second medical data by inputting the first medical data, the second medical data and the acquisition condition to a trained model.

### CROSS-REFERENCE TO RELATED APPLICATIONS

This application is based upon and claims the benefit of priority from Japanese Patent Application No. 2021-105899, filed Jun. 25, 2021, the entire contents of which are incorporated herein by reference.

### FIELD

Embodiments described herein relate generally to a medical information processing apparatus, a medical information processing method, and a medical information processing program.

### BACKGROUND

As the development of machine learning advances, employment of machine learning in, for example, a denoising process and a segmentation process has been advanced in medical fields as well. Since various observations are obtained by combining a plurality of items of imaging data with different characteristics, such as the combination of a magnetic resonance (MR) image acquired by a normal imaging method and a chemical shift image obtained by applying magnetic resonance spectroscopy (MRS) to a plurality of voxels, there is a need for applying machine learning to a combination of such items of imaging data.

However, since an MR image and a CSI image have different data characteristics, such as the difference in the resolution and the region of interest (ROI), such fluctuations in the characteristics of data may adversely affect training, thus causing deterioration of the properties of the trained model.

### BRIEF DESCRIPTION OF THE DRAWINGS

FIG. 1 is a block diagram showing a medical information processing apparatus according to an embodiment.

FIG. 2 is a diagram showing an example of an integrated network according to the embodiment.

FIG. 3 is a diagram showing an example of an integrated network which uses a multi-resolution neural network according to the embodiment.

FIG. 4 shows an example of a spectral distribution acquired in a CT apparatus according to the embodiment.

FIG. 5 is a diagram showing another example relating to first medical data and second medical data according to the embodiment.

### DETAILED DESCRIPTION

In general, according to one embodiment, a medical information processing apparatus comprising processing circuitry. The processing circuitry acquires first medical data having a first data resolution and second medical data having a second data resolution different from the first data resolution. The processing circuitry acquires an acquisition condition for medical data relating to at least one of the first medical data and the second medical data. The processing circuitry outputs a result integrated at least a piece of information that is based on the first medical data and the second medical data by inputting the first medical data, the second medical data and the acquisition condition to a trained model, the trained model being trained by input, as input data, a plurality of medical data items having different data resolutions and an acquisition condition for at least one of the plurality of medical data items, and input, as a correct data, a result obtained by integrating at least a piece of information that is based on the plurality of medical data items.

Hereinafter, a medical information processing apparatus, a medical information processing method, and a medical information processing program according to the present embodiment will be described with reference to the accompanying drawings. In the embodiments to be described below, elements assigned the same reference symbols are assumed to perform similar operations, and redundant descriptions will be omitted as appropriate. Hereinafter, an embodiment will be described with reference to the accompanying drawings.

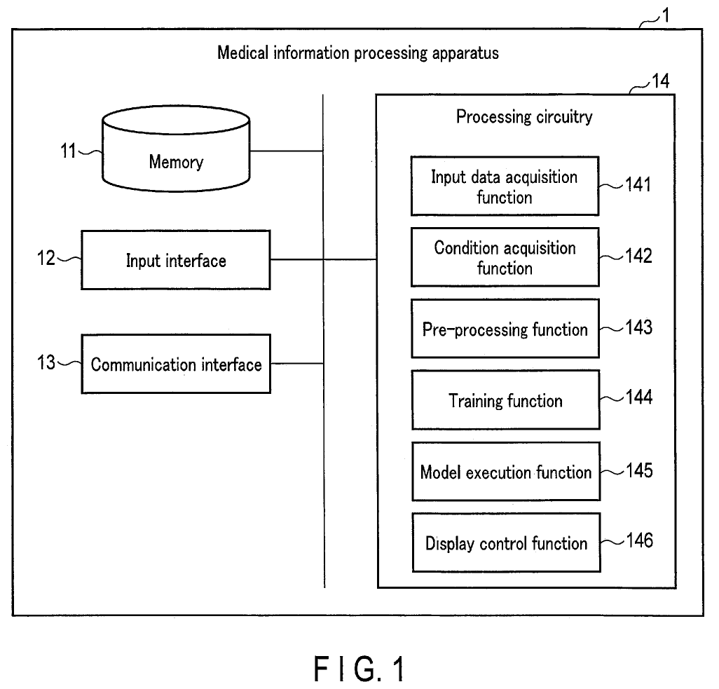

The medical information processing apparatus according to the present embodiment will be described with reference to the block diagram of FIG. 1.

A medical information processing apparatus 1 according to the present embodiment includes a memory 11, an input interface 12, a communication interface 13, and processing circuitry 14.

The medical information processing apparatus 1 according to the present embodiment may be included in a console, a workstation, etc., or may be included in a medical image diagnosis apparatus such as a magnetic resonance imaging (MRI) apparatus, a computed tomography (CT) apparatus, etc.

The memory 11 stores various types of medical data, acquisition conditions, trained models, etc., which will be discussed later. The memory 11 is a random-access memory (RAM), a semiconductor memory device such as a flash memory, a hard disk drive (HDD), a solid-state drive (SSD), an optical disk, or the like. The memory 11 may also be a drive or the like that reads and writes a variety of information from and to a portable storage medium such as a CD-ROM drive, a DVD drive, or a flash memory.

The input interface 12 includes circuitry that receives various instructions and information inputs from a user. The input interface 12 includes, for example, circuitry relating to a pointing device such as a mouse or an input device such as a keyboard. The circuitry included in the input interface 12 is not limited to circuitry relating to a physical operational component, such as a mouse or a keyboard. For example, the input interface 12 may include electric signal processing circuitry which receives an electric signal corresponding to an input operation from an external input device provided separately from the medical information processing apparatus 1, and outputs the received electric signal to a variety of circuits in the medical information processing apparatus 1.

The communication interface 13 executes data exchange with an external device in a wired or wireless manner. The description of the communication method and the structure of the interface will be omitted, since a common communication means may be used.

The processing circuitry 14 includes an input data acquisition function 141, a condition acquisition function 142, a pre-processing function 143, a training function 144, a model execution function 145, and a display control function 146. The processing circuitry 14 includes a processor (not illustrated) as a hardware resource.

The input data acquisition function 141 acquires first medical data and second medical data.

The first medical data is data having a first data resolution, and is, for example, a normal MR image acquired to obtain a contrast image (T1-weighted image, T2-weighted image, etc.) in an MRI apparatus. Herein, it is assumed that a “data resolution” refers to a unit data size such as a single voxel size or a duration (time width) in a timeline.

A case is assumed where the second medical data is data having a second data resolution, which is a data resolution different from the first data resolution, and is, for example, a chemical shift image obtained by the chemical shift imaging (CSI) technique, in which a magnetic resonance spectroscopy (MRS) image is acquired through a plurality of voxels. With a chemical shift image, it is possible to acquire information on metabolites in the body, unlike a normal MR image. As compared with a normal MR image, a chemical shift image has a low signal-to-noise ratio (SNR). While a single voxel of a normal MR image can be set to a voxel size of approximately “1×1×1 mm”, a single voxel of a chemical shift image is approximately “10×10×10 mm”, and has a low spatial resolution. A further difference is that an effective region of interest (ROI) relating to a chemical shift image is small, compared to a normal MR image. As described above, a case is assumed where the first medical data and the second medical data have different characteristics.

As will be described later, the first medical data may be a CT image, the second medical data may be a spectral CT image, the first medical data may be a CT image, and the second medical data may be a combination of a plurality of items of medical data acquired by different medical image acquiring apparatuses such as a chemical shift image, an ultrasonic image, a positron-emission tomography (PET) image, or a single-photon emission computed tomography (SPECT). That is, a combination of any type of medical data with different characteristics can be used for the combination of the first medical data and the second medical data.

The condition acquisition function 142 acquires a first acquisition condition corresponding to the first medical data and a second acquisition condition corresponding to the second medical data.

The first acquisition condition is a condition relating to an apparatus and a measurement target when the first medical data having a first data resolution has been acquired. For example, when the first medical data is an MR image, the first acquisition condition corresponds to imaging conditions such as parameters of an MRI apparatus when an MR image has been acquired, the measured portion of the subject, etc. More specifically, examples of the imaging conditions in an MRI apparatus include: imaging parameters such as the voxel size, the field of view (FoV), the ROI size, the matrix size, the echo time (TE), the repetition time (TR), the echo train length, the number of phase encoding steps, the flip angle, the collection time, a presence or absence of an inversion pulse, the number of slices, the slice thickness, the number of excitations (NEX), and the speed-up factor (acceleration factor) of parallel imaging; pulse sequence types relating to imaging such as the spin echo technique, the gradient echo technique, and the echo planar imaging (EPI) technique, and k-space trajectory types such as Cartesian scanning, radial scanning, and spiral scanning; and types of image reconstruction process such as the iterative reconstruction technique, the machine-learning reconstruction technique, etc.

The second acquisition condition is a condition relating to an apparatus when the second medical data has been acquired and to the measurement target. Specifically, when the second medical data is a chemical shift image, examples include the voxel size, the echo time (TE), the repetition time (TR), the number of additions, the spectral bandwidth, the sampling rate, and imaging sequence types such as the stimulated echo acquisition mode (STEAM) technique, the point-resolved stereoscopy (PRESS) technique, or the point-resolved stereoscopy echo-planar stereoscopic imaging (PRESS-EPSI) technique.

The pre-processing function 143 executes a data positioning process of the first medical data and the second medical data.

The training function 144 generates a trained model by learning information relating to the first medical data, the second medical data, and the acquisition condition in a shared manner in an integrated network including a first network that processes the first medical data and a second network that processes the second medical data. In other words, a trained model is a model to which a plurality of items of medical data having different data resolutions and an acquisition condition for at least one of the plurality of items of medical data are input, and which is trained to output a result obtained by integrating at least a piece of information that is based on the plurality of items of medical data.

The model execution function 145 inputs, to the trained model, first medical data and second medical data to be processed and an acquisition condition relating to at least one of the first medical data and the second medical data to be processed, and generates, as an inference result, a result obtained by integrating at least a piece of information that is based on the first medical data to be processed and a piece of information that is based on the second medical data to be processed.

The display control function 146 displays, for example, a training status of an integrated network by the training function 144 and a result of execution by the model execution function 145 on an external display, etc.

Various functions of the processing circuitry 14 may be stored in the memory 11 in the form of a program executable by a computer. In this case, the processing circuitry 14 can also be regarded as a processor which reads programs corresponding to these various functions from the memory 11 and executes the read programs to realize functions corresponding to the respective programs. In other words, the processing circuitry 14 that has read each program is equipped with the functions, etc. shown in the processing circuitry 14 of FIG. 1.

FIG. 1 illustrates the case where the various functions are realized in a single piece of processing circuitry 14; however, the processing circuitry 14 may be configured by a combination of a plurality of independent processors, and the functions may be realized by execution of the programs by the processors. In other words, each of the above-mentioned functions may be configured as a program, and a single piece of processing circuitry may execute each program, or a specific function may be implemented in exclusive, independent program-execution circuitry.

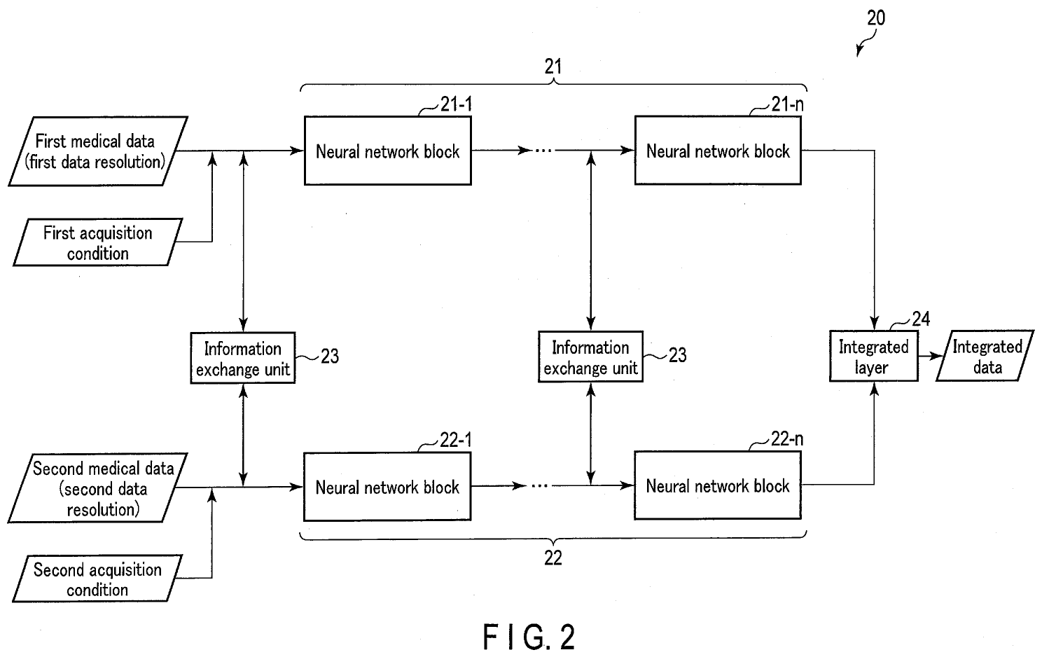

Next, an example of an integrated network of a training target according to the present embodiment will be described with reference to the conceptual diagram of FIG. 2.

An integrated network 20 according to the present embodiment includes a first network 21, a second network 22, one or more information exchange units 23, and an integrated layer 24.

Each of the first network 21 and the second network 22 includes a plurality of neural network blocks (hereinafter referred to as “NN blocks”). Specifically, the first network 21 includes n NN blocks 21-1 to 21-n (where n is a natural number equal to or greater than two), and the second network 22 similarly includes n NN blocks 22-1 to 22-n. An NN block is a type of function, and typically has parameters that can be learned.

Hereinafter, when the NN blocks 21-1 to 21-n of the first network 21 and the NN blocks 22-1 to 22-n of the second network are not distinguished from each other, each one of them will be simply referred to as an “NN block”.

It is assumed that an NN block is a deep-layer convolutional neural network in which a plurality of convolutional neural networks are concatenated; however, the configuration is not limited thereto, and any function or network structure used in machine learning may be adopted. The NN blocks in the first network 21 and those in the second network 22 may have either a common network structure or different network structures, or some of their network structures may be in common. For example, commonality of the network structure between the first network 21 and the second network 22 may be provided for each processing stage in the network depth direction in such a manner, for example, that the NN block 21-1 and the NN block 22-1 have a common network structure, and the NN block 21-2 and the NN block 22-2 have a common network structure. The first network 21 and the second network 22 may be or may not be configured of the same number of NN blocks.

First medical data having a first data resolution and a first acquisition condition relating to first medical data are input to the first network 21. Second medical data having a second data resolution and a second acquisition condition relating to second medical data are input to the second network 22.

Of the first medical data, the second medical data, the first acquisition condition, the second acquisition condition, a feature map produced in the process of data processing in the network, etc., the information exchange unit 23 exchanges one or more items of such information between the first network 21 and the second network 22. Specifically, one or more items of such information are input, and the input items of information themselves, or results obtained by applying some kind of function to them are used as a subsequent-stage input to at least one or more of the first network and the second network. Thereby, information can be shared between the first network 21 and the second network 22. The information exchange unit 23 may execute information exchange by inputting a copy of information to be exchanged to an opposite network, or the information exchange unit 23 may be configured of some kind of function, such as a convolutional neural network, and execute information exchange by inputting a feature amount of information to be exchanged to the opposite network.

For example, the first medical data and the first acquisition condition are input to the second network 22, and conversely, the second medical data and the second acquisition condition are input to the first network 21, and thereby information is exchanged. FIG. 2 shows an example in which the first acquisition condition and the second acquisition condition are respectively input to the opposite network sides by the information exchange unit 23 arranged at the head of the first network 21 and the second network 22; however, the configuration is not limited thereto. For example, the first acquisition condition and the second acquisition condition may not be input to an information exchange unit 23 located at the head of the network, and the first acquisition condition and the second acquisition condition may be input for the first time to an information exchange unit 23 at a later stage. The first acquisition condition and the second acquisition condition may be input to at least one or more of a plurality of information exchange units 23. That is, the same item of information may be input to a plurality of information exchange units 23.

It is assumed that the information exchange unit 23 is arranged at the stage prior to the input to each NN block between the first network 21 and the second network 22; however, there may be a location where the information exchange unit 23 is not arranged at the former stage of the input. Different acquisition conditions may be input to each information exchange unit 23. For example, information relating to the voxel size, the TE, and the TR may be input to the first-stage information exchange unit 23 as an acquisition condition, and the spectral bandwidth and the sampling rate may be input to the second-stage information exchange unit 23. The information exchange unit 23 may exchange information not only between the same processing stages but also between different processing stages of the first network 21 and the second network 22.

The integrated layer 24 generates integrated data by integrating an output of the first network 21 and an output of the second network 22, and outputs the generated integrated data to the outside. If, for example, the task of the integrated network 20 is tumor segmentation, an MR image and a chemical shift image may be input, and a superimposed image in which a tumor region is segmented may be output as integrated data. As the integrated data, a probability of a tumor as indicated by tumor-like characteristics may be output for each pixel. For example, when a probability of being a tumor of “0” is low and a probability of being a tumor of “1” is high, the probability of the tumor may be output for each pixel, with the probability of the pixel at the coordinate [10, 10] being “0.9” and the probability of the pixel at the coordinate [11, 11] being “0.2”. Alternatively, when, for example, the second medical data (e.g., a chemical shift image) has a relatively low resolution compared to the first medical data (e.g., an MR image), the second medical data (e.g., a chemical shift image) with a higher resolution may be used as an output. This is considered to be a kind of super-resolution. That is, any information can be output as the integrated data, if such data can be generated by data interpretation based on data with different characteristics.

Next, a method of inputting the first acquisition condition and the second acquisition condition to the integrated network 20 will be described.

When, for example, imaging conditions such as the ROT size, the TE, or the TR are input to the integrated network 20 as acquisition conditions at the time of collection of an MR image, since such imaging conditions are quantitative, the values of the parameters can be vectorized and input as they are. Alternatively, the values of the parameters can be normalized and input.

On the other hand, when an imaging condition is qualitative, such as the type of the imaging sequence, a vector representation digitized as the flag “0” or “1”, namely, what is known as “a one-hot vector”, can be input to the integrated network 20. Specifically, for example, when inputting to the integrated network 20 whether the spin echo technique or the gradient echo technique was used as the type of imaging sequence, [1, 0] is input in the case of the spin echo technique, and [0, 1] is input in the case of the gradient echo technique. Thereby, which of the techniques has been used can be represented as a flag. In this manner, a qualitative acquisition condition that needs to be input can be vectorized and input. The type of the medical data may be input as a one-hot vector. For example, if the medical data is an MR image, [1, 0] can be input, and if the medical data is a chemical shift image, [0,1] can be input.

Of course, a quantitative imaging condition and a qualitative imaging condition may be combined and input to the integrated network 20. In the case of an acquisition condition in which, for example, a weight λ1 is assigned to a qualitative condition “A” and a weight λ2 is assigned to a qualitative condition “B”, the presence or absence of “A” and “B” can be input as one-hot vectors, and the values themselves of the weights λ1 and λ2 can be input to the integrated network 20.

When the first medical data and the second medical data are input to the integrated network 20, adjustments may be performed to make the data resolutions uniform. It is assumed, for example, that an MR image of a voxel size of “1×1×1 mm” is input as the first medical data, and a chemical shift image of a voxel size of “10×10×10 mm” is input as the second medical data to the integrated network 20. The processing circuitry 14 may execute, through the pre-processing function 143, a conversion process of making the data resolutions uniform by cutting the second medical data into the voxel size of “1×1×1 mm”, and then the second medical data may be input to the integrated network 20. When the data resolutions of the first medical data and the second medical data differ in each axis of a three-dimensional space (an x-axis, a y-axis, and a z-axis), the data resolutions may be uniformly set to 1, or may be made uniform for each axis.

Instead of executing a conversion process of making the data resolutions uniform at the time of input of the second medical data, a conversion process of making the data resolutions uniform may be executed at a certain stage prior to integration of data in the integrated layer 24. If an MR image is input as the first medical data and the voxel size of “1×1×1 mm” of the MR image is input as the first acquisition condition, and a chemical shift image is input as second medical data and the voxel size of “10×10×10 mm” of the chemical shift image is input as the second acquisition condition, a conversion process of making the data resolutions uniform may be omitted. As the first acquisition condition and the second acquisition condition, a ratio of physical values relating to imaging conditions between the first medical data and the second medical data may also be used. When, for example, the voxel size of an MR image of the first medical data is “3×3×3 mm”, and the voxel size of an MR image of the second medical data is “10×10×10 mm”, the second acquisition condition may be a ratio such as “3/10”. If information relating to the data resolution is input to the integrated network 20 as the first acquisition condition and the second acquisition condition, training can be executed in consideration of the difference in data resolution.

The processing circuitry 14 may execute, through the pre-processing function 143, a data positioning process of the first medical data and the second medical data, and the data-positioned first medical data and second medical data may be input to the integrated network 20. For example, the processing circuitry 14 executes, through the pre-processing function 143, an image positioning (registration) process on an MR image and a chemical shift image. This allows the integrated network 20 to handle medical data.

(Training Process of Integrated Network)

Next, a training process of the integrated network 20 will be described.

When, for example, the integrated network 20 is employed as a classification task or a regression task during training of the integrated network 20, the processing circuitry 14 performs the training by training data through the training function 144 in such a manner that the first medical data, the second medical data, and at least one of the first acquisition condition and the second acquisition condition are used as input data, and the integrated result is used as correct data. For example, to minimize an error function relating to an error between an output of the integrated network 20 with respect to the input data and the correct data, parameters such as a weight, a bias, etc. of the integrated network 20 can be learned by what is known as “supervised learning” using a stochastic gradient descent, backpropagation, etc.

When, for example, the integrated network 20 is employed as a task for clustering, the first medical data, the second medical data, and at least one of the first acquisition condition and the second acquisition condition can be input to the integrated network 20, and the parameters of the integrated network can be learned according to the output of the integrated network 20 by what is known as “unsupervised learning”.

Through such training, a trained model of the integrated network 20 is generated.

The method of training the integrated network 20 is not particularly limited, and a training method that is commonly used in machine learning may be adopted according to the task to be solved by the integrated network 20.

(Inference Process of Integrated Network)

Next, an inference process using a trained model will be described.

Through a model execution function 145, the processing circuitry 14 inputs, to a trained integrated network which is a trained model, first medical data and second medical data to be processed, and a first acquisition condition corresponding to first medical data to be processed and a second acquisition condition corresponding to second medical data to be processed. Through processing of such data by a trained integrated network, a result obtained by integrating information relating to first medical data and second medical data to be processed is generated.

(Modification Example of Integrated Network)

In an integrated network, when what is known as a “multi-resolution neural network”, such as a U-net (see a non-patent document, “U-net: Convolutional Networks for Biomedical Image Segmentation” arXiv: 1505.04597) which is capable of handling multiple data resolutions, is used, since training and inference are executed in consideration of differences in data resolution, a conversion process by the pre-processing function 143 need not be executed. In a multi-resolution neural network, while a data resolution is lowered by a convolutional process by a downward path, a data resolution is increased by upsampling by an upward path, and thereby a plurality of resolutions can be handled. It is to be noted that any network structure capable of processing multiple resolutions, aside from a U-net, may be used.

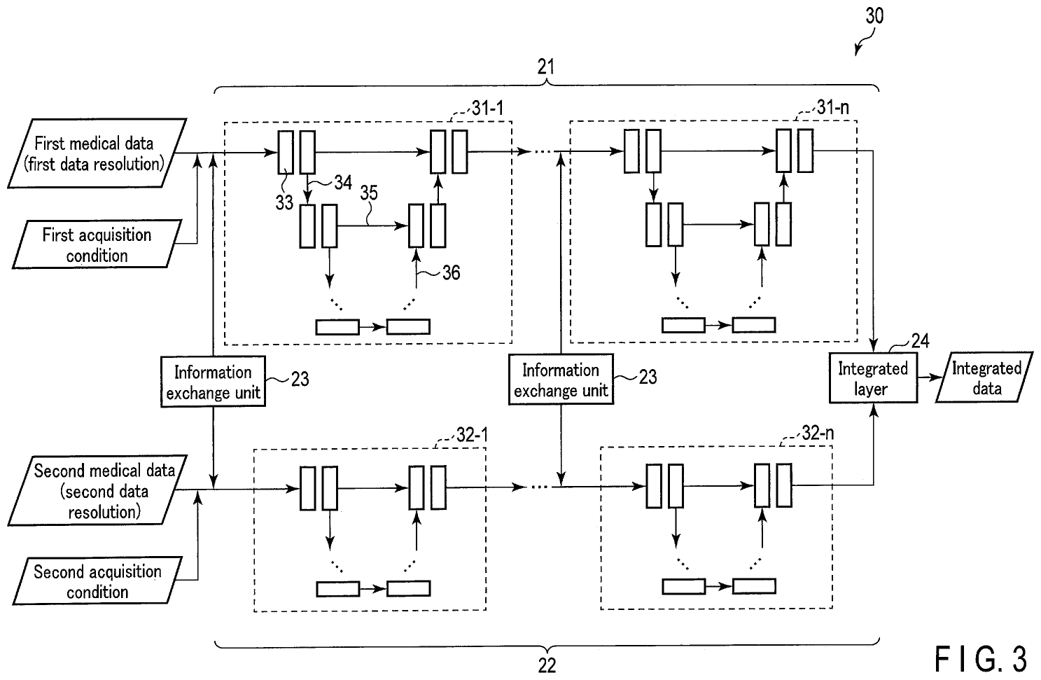

An example of an integrated network using a multi-resolution neural network will be described with reference to FIG. 3.

FIG. 3 shows an example in which an NN block shown in FIG. 2 is replaced with a block (hereinafter referred to as a “U-net block”) having a network structure of a U-net. Specifically, a first network 21 includes U-net blocks 31-1 to 31-n, and the first network 21 includes U-net blocks 32-1 to 32-n. Hereinafter, when reference is simply made to a “U-net block”, it refers to a U-net block included in both the first network 21 and the second network 22.

At the left side from the central axis, a U-net executes a plurality of stages of convolutional processes 33 and pooling processes 34 as processing of the encoder, and outputs a feature map at each stage. At the right side from the central axis, a U-net executes upsampling 36 and a convolutional process 33 while copying and concatenating a feature map which is an output from a layer of the same resolution at the left by a skip connection 35 as a decoder process. The network structure of the U-net is not limited to the example of FIG. 3, and a common U-net network structure may be used, with the number of the convolutional processes 33 at each stage, for example, being suitably set.

For a U-net block, the number of layers that process the same resolution, namely, the “depth” of the network is determined according to the data resolution of medical data to be input. That is, a U-net having a greater “depth” is used as the data resolution becomes higher, and a U-net with a smaller “depth” is used as the data resolution becomes lower. In the example of FIG. 3, a U-net block 31-1 that processes first medical data has a deeper network structure than a U-net block 32-1 that processes second medical data. The first medical data and the second medical data input to U-net block layers corresponding to the respective data resolutions produce an effect similar to that of a conversion process of making the data resolutions uniform.

The first acquisition condition, the second acquisition condition, and the processing of the information exchange unit 23 and the integrated layer 24 can be similarly applied as in the above-described case of FIG. 2, except for the difference in the position of input of the first medical data and the second medical data to the U-net block.

(Application to CT Data)

In the above-described example, MR data collected by an MRI apparatus such as an MR image and a chemical shift image has been assumed; however, imaging data of spectral CT such as dual energy CT, photon counting CT, K-edge imaging, etc. may be similarly processed by the integrated network 20. In spectral CT, data collection is executed using two types of energy spectra by switching between, for example, two types of tube voltages during imaging. Since an x-ray attenuation curve for a reference material is determined, material differentiation according to the type of atoms such as iodine and calcium can be realized through a computation based on a CT value obtained by the two types of energy spectra.

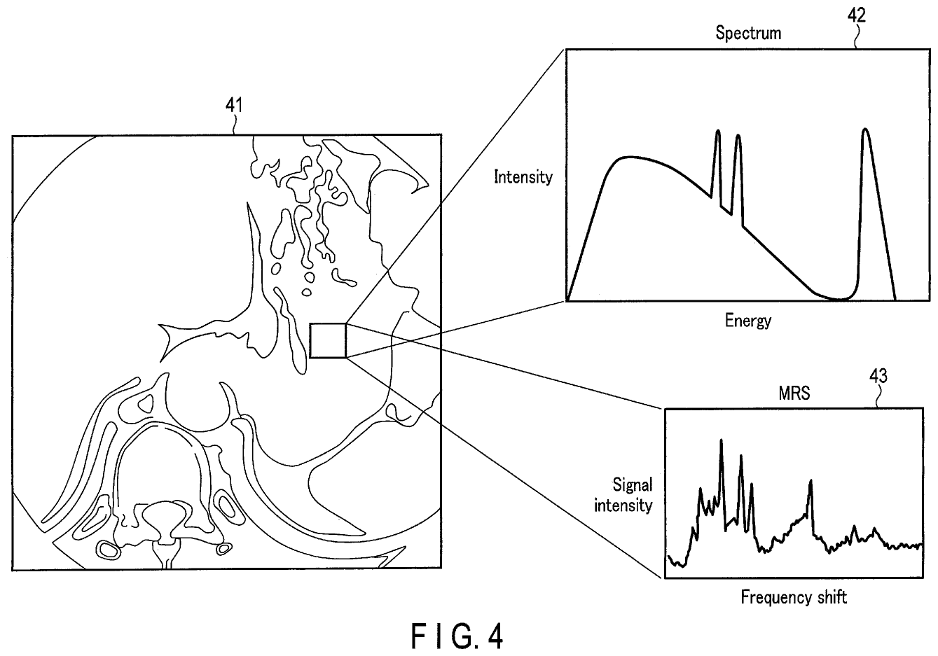

As an example, a spectral distribution acquired in a CT apparatus will be described with reference to FIG. 4.

FIG. 4 is an example of pixel information that configures an image 41. When the image 41 is a spectral CT image, a spectral distribution 42 showing a K-edge of a material contained in a tissue depicted in a pixel is obtained for each pixel. In the spectral distribution 42, the horizontal axis denotes an energy, and the vertical axis denotes an X-ray intensity. For comparison, when the image 41 is a chemical shift image, it has an MRS spectral distribution 43 for each pixel. In the spectral distribution 43, the horizontal axis denotes a frequency shift, and the vertical axis denotes a signal intensity.

In this manner, since the spectral distribution 42 of each pixel of a spectral CT image can be expressed in a similar manner to the spectral distribution 43 of each pixel of a chemical shift image, by inputting, to the integrated network 20, the first medical data as a normal CT image and the second medical data as a spectral CT image, they can be treated in a manner similar to MR data.

In particular, a spectral CT image acquired by photon counting CT, for which energy differentiation is performed for individual photons, is considered to have a low SNR and a low partial resolution, compared to a normal CT image. Accordingly, when a normal CT image is used as the first medical data and a photon counting CT image with a low data resolution is used as the second medical data, they have a relationship similar to the case where the first medical data is an MR image and the second medical data is a chemical shift image.

As an acquisition condition for CT data, for example, an imaging parameter of an X-ray CT apparatus such as the tube current, the tube voltage, the slice thickness, or the type of energy distribution, the type of image reconstruction process such as the filtered back projection

(FBP) technique or the iterative reconstruction technique, and whether the image is a normal CT image or a spectral CT image may be represented in a one-hot vector and input to the integrated network 20. An energy distribution of each pixel may be input in the form of a waveform.

The first medical data may be an MR image, the second medical data may be a spectral CT image, the first medical data may be a spectral CT image, the second medical data may be a chemical shift image, acquisition conditions of various types of images may be input to the integrated network 20, and a training process and an inference process may be executed.

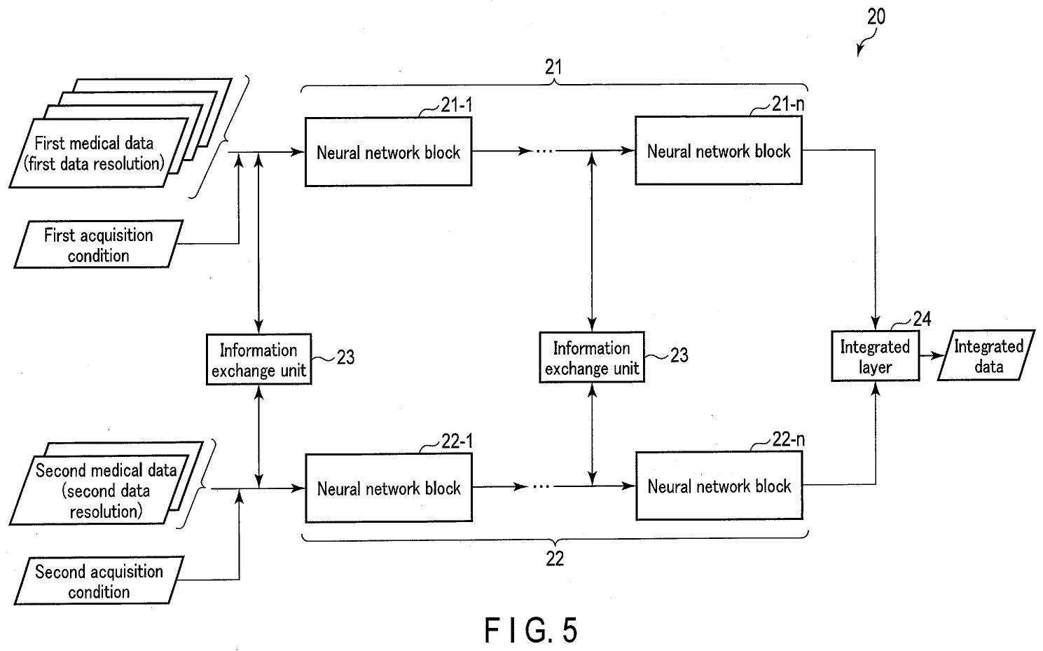

Next, another example of input of the first medical data and the second medical data will be described with reference to FIG. 5.

In the above-described example, a difference in unit data size such as a voxel size is assumed as a difference in data resolution; however, the configuration is not limited thereto, and a duration, for example, may be a data resolution.

The first medical data shown in FIG. 5 is time-series data acquired in a duration, and the second medical data is time-series data in a duration shorter than that of the first medical data. For example, a plurality of MR images acquired over a couple of months for follow-up and imaging conditions relating to these MR images are input as first medical data and as a first acquisition condition, respectively, to the integrated network 20. On the other hand, by inputting, to the integrated network 20, a single MR image that has been acquired most recently and an imaging condition relating to the single MR image as second medical data, a training process and an inference process of the integrated network 20 can be performed.

When a plurality of items of first medical data or a plurality of items of second medical data are input to the integrated network 20, the processing circuitry 14 may perform, through the pre-processing function 143, a conversion process of making the plurality of items of first medical data uniform or making the plurality of items of second medical data uniform. For example, a normalization may be performed in the case of spectral data, and a registration process may be performed in the case of an image.

One of the first medical data and the second medical data may be partly missing data. When partly missing data is handled during training of the integrated network 20, incomplete data may be generated by executing a random mask process, in which a randomly generated mask is applied to complete data with no missing data, and the generated incomplete data with some missing data may be used as training data. The integrated network 20 can be trained by inputting thereto information of the part missing in the mask process, along with the generated incomplete data, as an acquisition condition.

Specifically, it is assumed, for example, that incomplete data obtained by applying a mask process to a complete MR image to cause a partial region to be missing is used as the first medical data. The integrated network 20 can be trained by using, as input data, the missing MR image and the first acquisition condition including coordinate information of the region that has been caused to be missing by the mask process. Instead of the coordinate information of the missing region, mask information relating to the shape and size of the mask used in a mask process may be used.

During inference, medical data can be processed in a manner similar to the inference process by inputting, to the trained model of the integrated network 20 that has been trained using incomplete data, as described above, a partly missing MR image as first medical data and coordinate information relating to the missing region as a first acquisition condition. Of course, even when the second medical data is partly missing data, it can be processed in a manner similar to the case where part of the first medical data is missing.

Furthermore, the configuration is not limited to the case where data is partly missing, and even low-reliability data with a low data reliability can be handled in a similar manner. For example, a positioning image acquired by positioning scanning in a CT apparatus may be referred to as low-reliability data which has a lower image reliability than a CT image acquire by the scanning described herein. Accordingly, training of the integrated network 20 and inference based on a trained model may be executed using low-reliability data of a positioning image as first medical data and a reliability of a positioning image as a first acquisition condition. As the reliability of a positioning image, for example, a ratio of a resolution of a CT image to a resolution of a positioning image may be used.

According to the present embodiment described above, first medical data and second medical data having different data resolutions, and at least one of a first acquisition condition of first medical data and a second acquisition condition of second medical data are input to an integrated network, and training and inference are performed. By thus inputting, to the integrated network, acquisition conditions of medical data in addition to the medical data, training can be executed by sharing medical data and information relating to acquisition conditions in view of fluctuations relating to conditions of a plurality of items of medical data with low data resolutions, namely, the adverse effects of statistic characteristics of data, thus reducing adverse effects caused by characteristics of data. It is thereby possible to improve the precision in training, thus improving the properties of the trained model.

According to at least one embodiment described above, it is possible to reduce adverse effects caused by characteristics of data.

Furthermore, the functions described in connection with the above embodiment may be implemented, for example, by installing a program for executing the processing in a computer, such as a workstation, etc., and expanding the program in a memory. The program that causes the computer to execute the above-described technique can be stored and distributed by means of a storage medium, such as a magnetic disk (a hard disk, etc.), an optical disk (CD-ROM, DVD, etc.), and a semiconductor memory.

Herein, the term “processor” used in the above description means, for example, circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), or a field-programmable gate array (FPGA).

While certain embodiments have been described, these embodiments have been presented by way of example only, and are not intended to limit the scope of the inventions.

Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing apparatus comprising processing circuitry configured to:
acquire first medical data having a first data resolution and second medical data having a second data resolution different from the first data resolution;
acquire an acquisition condition for medical data relating to at least one of the first medical data and the second medical data; and
output a result integrated at least a piece of information that is based on the first medical data and the second medical data by inputting the first medical data, the second medical data and the acquisition condition to a trained model, the trained model being trained by input, as input data, a plurality of medical data items having different data resolutions and an acquisition condition for
at least one of the plurality of medical data items, and input, as a correct data, a result obtained by integrating at least a piece of information that is based on the plurality of medical data items.

2. The medical information processing apparatus according to claim 1, wherein
the first medical data and the second medical data have different unit data sizes.

3. The medical information processing apparatus according to claim 1, wherein
at least one of the first medical data and the second medical data is time-series data, and
the first medical data and the second medical data are acquired in different durations in a timeline.

4. The medical information processing apparatus according to claim 1, wherein
the first medical data is a magnetic resonance (MR) image,
the second medical data is a chemical shift image, and
the acquisition condition is an imaging condition relating to at least one of the MR image and the chemical shift image.

5. The medical information processing apparatus according to claim 1, wherein
the first medical data is a computed tomography (CT) image,
the second medical data is a spectral CT image, and
the acquisition condition is an imaging condition relating to at least one of the CT image and the spectral CT image.

6. The medical information processing apparatus according to claim 1, wherein
the first medical data is data acquired by a magnetic resonance imaging (MRI) apparatus,
the second medical data is data acquired by an X-ray computed tomography (CT) apparatus, and
the acquisition condition is an imaging condition relating to at least one of the MRI apparatus or the X-ray CT apparatus.

7. The medical information processing apparatus according to claim 1, wherein
the acquisition condition is a condition relating to at least one of an imaging parameter, a pulse sequence, and a type of image reconstruction process.

8. The medical information processing apparatus according to claim 1, wherein
the acquisition condition includes a ratio of physical values relating to imaging conditions between the first medical data and the second medical data.

9. The medical information processing apparatus according to claim 1, wherein the processing circuitry further configured to generate a result in which data having a relatively low data resolution among one of the first medical data and the second medical data is integrated as data having a higher data resolution.

10. A medical information processing method, comprising:
acquiring first medical data having a first data resolution and second medical data having a second data resolution different from the first data resolution;
acquiring an acquisition condition for medical data relating to at least one of the first medical data and the second medical data; and
outputting a result integrated at least a piece of information that is based on the first medical data and the second medical data by inputting the first medical data,
the second medical data and the acquisition condition to a trained model, the trained model being trained by input, as input data, a plurality of medical data items having different data resolutions and an acquisition condition for at least one of the plurality of medical data items, and input, as a correct data, a result obtained by integrating at least a piece of information that is based on the plurality of medical data items.

11. A medical information processing apparatus comprising processing circuitry configured to:
acquire first medical data having a first data resolution and second medical data having a second data resolution different from the first data resolution;
acquire an acquisition condition for medical data relating to at least one of the first medical data and the second medical data; and
generate a trained model by training an integrated network with sharing information relating to the first medical data, the second medical data, and the acquisition condition, the integrated network including a first network that processes the first medical data and a second network that processes the second medical data.

12. The medical information processing apparatus according to claim 11, wherein
the integrated network includes an information exchange unit which exchanges information relating to the first network and information relating to the second network.

13. The medical information processing apparatus according to claim 11, wherein
the integrated network includes a plurality of information exchange units, and
the processing circuitry trains the integrated network by inputting the acquisition condition to at least one of the information exchange units.
